(19) United States
(12) Patent Application Publication
(10) Pub. No.: US 2016/0074458 A1
(43) Pub. Date: Mar. 17, 2016

(54) COMPOSITION FOR PREVENTION AND TREATMENT OF MILIARIA

(71) Applicant: Weiyao Shi, Beijing (CN)

(72) Inventor: Weiyao Shi, Beijing (CN)

(21) Appl. No.: 14/486,048

(22) Filed: Sep. 15, 2014

(51) Int. Cl. A61K 36/489; A61K 36/36; A61K 36/904; A61K 36/756; A61K 36/35; A61K 36/28; A61K 31/125

(52) U.S. Cl. CPC A61K 36/489; A61K 36/28; A61K 36/36; A61K 31/125; A61K 36/756; A61K 36/35; A61K 36/904

(57) ABSTRACT

A composition for prevention and treatment of miliaria is disclosed by use of a purslane extract with or without a stevia extract.

### FIELD OF THE INVENTION

The present invention relates to a traditional Chinese medicinal composition for prevention and treatment of miliaria.

### BACKGROUND OF THE INVENTION

Miliaria is a skin disease marked by small and itchy rashes. Miliaria is a common ailment in hot and humid conditions, such as in the tropics and during the summer season. Although it affects people of all ages, it is especially common in children and infants due to their underdeveloped sweat glands.

In China, many traditional Chinese medicinal compositions have been developed for the treatment of miliaria for many years. For example, CN201310698403 discloses a traditional Chinese medicinal composition for the treatment of miliaria, comprising several traditional Chinese medicines.

However, there is a continuous need for improved traditional Chinese medicinal compositions for the treatment of miliaria.

### DESCRIPTION OF THE INVENTION

The invention provides a traditional Chinese medicinal composition used for the prevention and treatment of miliaria. Preferably, the composition is in powder form.

The traditional Chinese medicinal composition comprises several traditional Chinese medicines that are conventionally used for the treatment of miliaria, characterized in that it additionally comprises purslane extract and/or stevia extract.

The present invention is based on a conventional traditional Chinese medicinal composition for the treatment of miliaria, e.g. that disclosed in CN201310698403, comprising Pollen Pini, purslane, mentha-camphor, Borneolum Syntheticum (borneol), Indian fig (cactus) powder, Radix Glycyrrhizae (liquorice root), diatomaceous earth, Radix Stemonae (stemona), calamina, Radix Sophorae Flavescentis (sophora), Fructus Forsythiae (forsythia), Cortex Phellodendri (golden cypress), and camphor.

The inventor of the present invention finds that, surprisingly, by replacing the purslane in the traditional Chinese medicinal composition for the treatment of miliaria with purslane extract, the efficiency of the composition in treating miliaria can be significantly improved.

Furthermore, in addition to or alternative to purslane extract, stevia extract is incorporated into the composition for improving the efficiency of the composition in the treatment of miliaria.

Purslane, also known as Portulaca oleracea or “Ma Chi Xian”, has been used as traditional Chinese medicine for many years. Its leaves are conventionally used for the treatment of skin diseases.

The inventor of the present invention finds that, surprisingly, by enriching the active components in purslane by extracting, the efficiency of the composition in treating miliaria can be significantly improved.

As used herein, the term “purslane extract” is intended to mean an extract obtainable from a purslane plant. In a preferred aspect of the present invention, the purslane extract is water extract of purslane.

Stevia is a genus of about 240 species of herbs and shrubs in the sunflower family (Asteraceae), native to subtropical and tropical South America and Central America.

The species Stevia rebaudiana Bertoni, commonly known as sweet leaf, sugarleaf, or simply stevia, is widely grown for its sweet leaves. The leaves have traditionally been used as a sweetener. Steviosides and rebaudiosides are the major constituents of glycosides found in the leaves of the stevia plant. Although conventionally being used as a sweetener, it is also disclosed that stevia is particularly advantageous for preventing or treating disorders related to skin aging.

The inventor of the present invention finds that, surprisingly, in addition to the advantageous effects on skin aging, stevia extract also has advantageous effects on the prevention and treatment of miliaria, such effects are unexpectable.

Furthermore, the inventor of the present invention also finds that, surprisingly, stevia extract has synergistic effects with purslane extract in the treatment of miliaria.

As used herein, the term “stevia extract” is intended to mean an extract obtainable from a stevia plant. In a preferred aspect of the present invention, the stevia extract is water extract of stevia.

Therefore, in an aspect, the invention provides a traditional Chinese medicinal composition used for the prevention and treatment of miliaria, comprising conventional traditional Chinese medicines for the prevention and treatment of miliaria, characterized in that it comprises purslane extract and stevia extract.

In a preferred aspect, the invention provides a traditional Chinese medicinal composition used for the prevention and treatment of miliaria, comprising Pollen Pini, mentha-camphor, Borneolum Syntheticum (borneol), Indian fig (cactus) powder, Radix Glycyrrhizae (liquorice root), diatomaceous earth, Radix Stemonae (stemona), calamina, Radix Sophorae Flavescentis (sophora), Fructus Forsythiae (forsythia), Cortex Phellodendri (golden cypress), and camphor, characterized in that it additionally comprises purslane extract and/or stevia extract.

In a more preferred embodiment of the present invention, the traditional Chinese medicinal composition according to the present invention comprises (by weight parts): Pollen Pini from about 10 to about 20, mentha-camphor from about 5 to about 10, Borneolum Syntheticum (borneol) from about 5 to about 10, Indian fig (cactus) powder from about 1 to about 10, Radix Glycyrrhizae from about 20 to about 30, diatomaceous earth from about 10 to about 20, Radix Stemonae from about 5 to about 20, calamina from about 5 to about 20, Radix Sophorae Flavescentis from about 10 to about 15, Fructus Forsythiae from about 10 to about 20, Cortex Phellodendri from about 10 to about 20, camphor from about 10 to about 20, characterized in that it additionally comprises purslane extract from about 10 to about 50 and/or stevia extract from about 1 to about 50.

By replacing purslane in traditional Chinese medicine with purslane extract, the inventors find that, surprisingly, the efficiency of the composition in treating miliaria can be significantly improved.

By incorporating stevia extract into the formula of a conventional traditional Chinese medicinal composition, the inventors find that, surprisingly, the effects of the medicinal composition on the prevention and treatment of miliaria is also significantly improved.

The inventors also find that, surprisingly, there is a synergistic effect between purslane extract and stevia extract.

The invention will be further described with reference to the following non-limiting Examples. It will be apparent to those skilled in the art that many changes can be made in the embodiments described without departing from the scope of the present invention. Thus the scope of the present invention should not be limited to the embodiments described in this application, but only by embodiments described by the language of the claims and the equivalents of those embodiments. Unless otherwise indicated, all percentages are by weight.

### Examples

### Example 1

### Formulation of the Traditional Chinese Medicinal Composition for Treating Miliaria

The compositions of the samples of the present invention and a control sample were formulated according to the following Table 1.

All of the traditional Chinese medicines, including Pollen Pini, purslane, mentha-camphor, Borneolum Syntheticum, Indian fig powder, Radix Glycyrrhizae, diatomaceous earth, Radix Stemonae, calamina, Radix Sophorae Flavescentis, Fructus Forsythiae, Cortex Phellodendri, and camphor, were obtained from a commercial source, Tongrentang Pharmacy.

The purslane extract was obtained according to the process described in CN201301537803.6. i.e. by pulverizing purslane raw materials, extracting with purified water (available from Wahaha) at weight ratio of 1:5 at 100° C. for 3 h, filtering with filter paper, and then drying the filtrate.

The stevia extract was obtained by chopping dried stevia leaves into pieces, and extracting with purified water (available from Wahaha) at weight ratio of 1:5 at 70° C. for 6 h, filtering with filter paper, and then drying the filtrate.

The components were mixed together and then processed into dusting powder forms.

TABLE 1
Traditional Chinese Medicinal Composition
for Prevention and Treatment of Miliaria

| | | | | Control |
|---|---|---|---|---|
| | Example 1 | Example 2 | Example 3 | Example |
| Pollen Pini | 15 | 15 | 15 | 15 |
| mentha-camphor | 5 | 5 | 5 | 5 |
| Borneolum | 5 | 5 | 5 | 5 |
| Syntheticum | | | | |
| Indian fig | 10 | 10 | 10 | 10 |
| (powder) | | | | |
| Radix | 20 | 20 | 20 | 20 |
| Glycyrrhizae | | | | |
| diatomaceous | 15 | 15 | 15 | 15 |
| earth | | | | |
| Radix Stemonae | 20 | 20 | 20 | 20 |
| calamina | 20 | 20 | 20 | 20 |
| Radix Sophorae | 10 | 10 | 10 | 10 |
| Flavescentis | | | | |
| Fructus | 10 | 10 | 10 | 10 |
| Forsythiae | | | | |
| Cortex | 10 | 10 | 10 | 10 |
| Phellodendri | | | | |
| camphor | 20 | 20 | 20 | 20 |
| Stevia leaves | 50 | — | — | — |
| (dried, pulverized) | | | | |
| Purslane | — | | — | 30 |
| Purslane extract | 30 | — | 30 | — |
| Stevia extract | — | 30 | 30 | — |

### Example 2

### Effects of the Composition of the Present Invention on the Prevention and Treatment of Miliaria

Five subjects with miliaria were treated with the compositions of examples 1-3 and the control sample. For each subject, the dusting powder of all of the four compositions were simultaneously and topically applied onto distinct areas affected with miliaria rash/blisters on the skin of neck and back by using cotton pads. The effects of the four compositions on miliaria were evaluated for seven days.

The results showed that, the composition comprising either purslane extract (Example 1) or stevia extract (Example 2) had better performances on the treatment of miliaria, i.e. the symptom of miliaria in the areas being treated with the such compositions were relieved faster, in comparison to that being treated with the composition of control sample.

The results also showed that, the composition comprising both purslane extract and stevia extract (Example 3) had even better performances on the treatment of miliaria, demonstrating the synergistic effect between purslane extract and stevia extract.

1. A composition used for the prevention and treatment of miliaria, comprising Pollen Pini, mentha-camphor, Borneolum Syntheticum (borneol), Indian fig (cactus) powder, Radix Glycyrrhizae, diatomaceous earth, Radix Stemonae, calamina, Radix Sophorae Flavescentis, Fructus Forsythiae, Cortex Phellodendri, and camphor, characterized in that it additionally comprises purslane extract and/or stevia extract.

2. The composition according to claim 1, comprising, by weight parts: Pollen Pini from about 10 to about 20, mentha-camphor from about 5 to about 10, Borneolum Syntheticum (borneol) from about 5 to about 10, Indian fig (cactus) powder from about 1 to about 10, Radix Glycyrrhizae from about 20 to about 30, diatomaceous earth from about 10 to about 20, Radix Stemonae from about 5 to about 20, calamina from about 5 to about 20, Radix Sophorae Flavescentis from about 10 to about 15, Fructus Forsythiae from about 10 to about 20, Cortex Phellodendri from about 10 to about 20, camphor from about 10 to about 20, characterized in that it additionally comprises purslane extract from about 10 to about 50 and/or stevia extract from about 1 to about 50.

3. The composition according to claim 1, wherein the purslane extract is water extract of purslane.

4. The composition according to claim 2, wherein the purslane extract is water extract of purslane.

5. The composition according to claim 1, wherein the stevia extract is water extract of stevia.

6. The composition according to claim 2, wherein the stevia extract is water extract of stevia.
